(19) United States
(12) Patent Application Publication
(10) Pub. No.: US 2018/0078217 A1
(43) Pub. Date: Mar. 22, 2018

(54) METHOD AND APPARATUS FOR PROVIDING INFORMATION TO A PATIENT DURING A MEDICAL IMAGING EXAMINATION THAT INDICATES THE RESIDUAL EXAMINATION TIME FOR THE EXAMINATION

(71) Applicant: Siemens Healthcare GmbH, Erlangen (DE)

(72) Inventors: Stephan Nufer, Erlangen (DE); Anja Kuerten, Obermichelbach (DE); Lea Schubart, Erlangen (DE); Jens Thoene, Lauf an der Pegnitz (DE)

(73) Assignee: Siemens Healthcare GmbH, Erlangen (DE)

(21) Appl. No.: 15/709,762

(22) Filed: Sep. 20, 2017

(30) Foreign Application Priority Data
Sep. 20, 2016 (DE) 102016218009.6

(51) Int. Cl. A61B 5/00; G07C 3/04

(52) U.S. Cl. CPC A61B 5/743; G07C 3/04; A61B 5/7415

(57) ABSTRACT

A method for providing information to a patient during a medical imaging examination, said method comprising: operating a medical imaging apparatus, while a patient is undergoing an examination with the medical imaging apparatus, during said medical imaging examination, providing a computer with time information that informs said computer as to an amount of time that has elapsed from a beginning of said medical imaging examination, from said time information, determining, in said computer, a residual examination time for the medical imaging examination that designates an ending time at which said medical imaging examination is expected to end, generating output information in said computer dependent on said residual examination time, said output information comprising humanly perceptible information formatted to inform the patient as to when said medical imaging examination is expected to end; and providing said output information in said humanly perceptible form to the patient while the patient is undergoing said medical imaging examination.

### BACKGROUND OF THE INVENTION

### Field of the Invention

The present invention relates to a method, a medical imaging apparatus, and a non-transitory data storage medium for providing output information to a patient during a medical imaging examination, wherein the information indicates the residual examination time for the medical imaging examination.

### Description of the Prior Art

Medical imaging examinations of a patient can take varying lengths of time depending on the type of the medical imaging examination and/or depending on the body region of the patient and/or depending on the medical and/or diagnostic issue in question. An examination can take from a few minutes to an hour or longer. Particularly for magnetic resonance examinations, it is usual for examinations to take an hour and longer.

Patients frequently find it difficult to estimate the remaining examination time correctly during a medical imaging examination. This can cause a patient to experience uncertainty or a state of anxiety. It can also result in unwanted interruptions to medical imaging examinations causing further delays to the medical imaging examination.

### SUMMARY OF THE INVENTION

An object of the present invention is to provide time information to a patient about the remaining residual examination time for a medical imaging examination on the patient.

This object is achieved by a method for providing such output information to a patient during a medical imaging examination, having the following steps. At least one item of time information for the medical imaging examination is acquired. The residual examination time for the medical imaging examination is computed by a processor using the time information. Output information are generated by the processor as a function of the residual examination time. The output information is provided to the patient in a way that is perceptible by the patient in the imaging apparatus during the examination.

The medical imaging examination is performed by a medical imaging apparatus, which can be a magnetic resonance apparatus, a computed tomography apparatus, a PET apparatus (positron emission tomography apparatus) and/or a further medical imaging apparatus that appear advisable to those skilled in the art. Consequently, the medical imaging examination can be a magnetic resonance examination, a computed tomography examination, a PET examination etc. The medical imaging examination preferably is a magnetic resonance examination since, in this case, the method according to the invention can be used particularly advantageously due to the long examination times.

The acquisition of the at least one item of time information is preferably performed by a time-acquisition unit. The time-acquisition unit can be part of a control computer of the medical imaging apparatus. The time-acquisition unit can also be embodied separately from the control computer of the medical imaging apparatus. The acquisition of the time information is preferably performed automatically and/or autonomously by the time-acquisition unit.

The time information preferably is information relating to a time profile of the medical imaging examination. The time information is based on current information and/or a current time point and/or current time, and a calculation as to how long the medical imaging examination has already been performed on the patient from the beginning of the medical imaging examination to the time point of the acquisition of the time information.

The residual examination time for the medical imaging examination is the anticipated residual examination time for the medical imaging examination. The anticipated residual examination time is a time and/or a duration of a remaining duration of the medical imaging examination at the time point of the acquisition of the time information. Accordingly, the residual examination time, in particular the anticipated residual examination time, indicates how long the medical imaging examination will last from the time point of the acquisition of the time information to its end. Hence, as the medical imaging examination progresses, the residual examination time, in particular the anticipated residual examination time, has a steadily decreasing time value. The residual examination time, in particular the anticipated residual examination time, for the medical imaging examination is preferably established using the time information, in particular using the current information and/or using the current time point and/or using the current time, how long the medical imaging examination has already been performed from the beginning of the medical imaging examination on the patient.

The establishment of the residual examination time is preferably performed by the control computer. The control unit can be part of the medical imaging apparatus. To this end, the control computer preferably has computer programs and/or software that establish the residual examination time for the medical imaging examination using the time information when executed on a processor of the control unit. The control computer preferably executes an evaluation algorithm and/or determining algorithm for the establishment of the residual examination time for the medical imaging examination.

The output information is generated by the control computer using computer programs and/or software, which generate the output information as a function of the residual examination time when executed by a processor of the control unit. The control computer preferably comprises an evaluation algorithm and/or determining algorithm to generate the output information as a function of the residual examination time.

The output information can be optical output information and/or acoustic output information. The acoustic output information is preferably provided by headphones worn by the patient during the medical imaging examination and/or by a speaker system.

The optical output information can be provided by an optical output unit, such as a monitor and/or a screen situated inside a patient-receiving region of the medical imaging apparatus. The optical output information can also be provided by a projection device that projects the optical output information onto a projection surface in the visual range of the patient. Optical output information originating from outside the patient-receiving region of the medical imaging apparatus is projected onto a projection surface inside the patient-receiving region. The optical output information can also be provided by video glasses worn by the patient during the medical imaging examination. It is also possible for optical output information to be provided by a lighting unit of the medical imaging apparatus for lighting and/or illuminating the patient-receiving region.

The output information, in particular the optical and/or acoustic output information, preferably includes information relating to the anticipated residual examination time.

The invention enables an advantageous communication of information to the patient during the medical imaging examination without having to interrupt the medical imaging examination. This is in turn results in timesaving communication of information to the patient during the medical imaging examination. The output information during the medical imaging examination can also give the patient a temporal pointer for categorizing the current situation in terms of time. This can advantageously help to relax the patient so that delays and/or repetitions of individual scans or partial scans in the medical imaging examination due to a nervous patient can be avoided.

The method for providing the output information for the medical imaging examination, wherein the output information indicates the residual examination time for the medical imaging examination for the patient, is preferably performed automatically and/or autonomously by means of the control computer of the medical imaging apparatus. This has the advantage that it enables a particularly timesaving examination procedure to be achieved. In particular, a medical operator carrying out the medical imaging examination, such as, for example, a doctor or a radiographer can concentrate on the performance of the medical imaging examination since there is no need for the medical operator to monitor the time and communicate time information manually.

In an embodiment of the invention, the at least one item of time information designates a duration of the medical imaging examination from the beginning of the medical imaging examination until the acquisition of the at least one item of time information for the medical imaging examination. This enables particularly simple acquisition of the time information, which can be acquired with the use of conventional time-acquisition units, such as a clock. This also enables the provision of a particularly timesaving determination of the output information since the use of such time information enables the anticipated residual examination time and hence also the output information to be determined directly.

In a further embodiment of the invention, the residual examination time for the medical imaging examination is established in comparison to a total duration of the medical imaging examination. A total duration of the medical imaging examination means a duration of the medical imaging examination for which the medical imaging examination lasts from the beginning of the medical imaging examination to an end, in particular a planned end, of the medical imaging examination. This enables a simple establishment and/or determination of the residual examination time, in particular the anticipated residual examination time, for the medical imaging examination using the established time information and the total duration of the medical imaging examination.

The total duration of the medical imaging examination can be calculated by the control computer using settings for the medical imaging examination. This enables a particularly exact determination and/or establishment of the total duration of the medical imaging examination and hence the anticipated residual examination time for the medical imaging examination. The settings of the medical imaging examination are dependent on the type of the medical imaging examination, for example whether the medical imaging examination is a magnetic resonance examination or a computed tomography examination etc. The settings of the medical imaging examination can also be dependent on an examination region of the patient, such as a head or a knee or an abdomen, etc. The settings of the medical imaging examination can also be dependent on a medical or diagnostic issue. The settings of the medical imaging examination can be parameter settings that influence the duration of the medical imaging examination. For a magnetic resonance examination, the parameter settings can be, for example, an echo time and/or a slice thickness etc. Further settings of the medical imaging examination that appear advisable to those skilled in the art are also possible.

In a further embodiment of the invention, the total duration of the medical imaging examination is established as a function of an empirical value. The empirical value can be provided manually and/or entered by a medical operator carrying out and/or planning the medical imaging examination, for example a doctor. For example, the total duration of an abdominal examination can last several minutes longer than established by the control computer using the settings of the medical imaging examination. The causes of this can be, for example, breathing instructions to the patient, changes in the position of the patient table and/or pauses for contrast agent accumulations, wherein such causes are not originally taken into account in the calculation of the total duration by the control computer using the settings of the medical imaging examination. Particularly in the case of breathing-triggered medical imaging examinations, the total duration of medical imaging examinations is also dependent upon the patient's breathing rate and/or breathing amplitude, which as such are not taken into account in the calculation of the total duration by means of the control computer.

This embodiment of the invention enables an estimation that is as realistic as to the anticipated residual examination time, and hence the output information. In addition, this does not cause the patient to become confused during the medical imaging examination due to incorrect output information, such as output information announcing the end of the medical imaging examination when this cannot yet be predicted due to the delays. In addition, this enables output information to be provided at the beginning of a medical imaging examination, even if the planning of the medical imaging examination has not yet been fully completed and hence the specific determination of the anticipated residual examination time is not yet possible.

It is also possible to provide several items of output information, which are generated during the medical imaging examination. The several items of output information indicate a time profile for the patient. The several items of output information are preferably generated in succession in terms of time at different time points of the medical imaging examination, and hence also provided to the patient as respective outputs in succession in terms of time at different time points of the medical imaging examination. The several items of output information provided in succession in terms of time at different time points of the medical imaging examination can be used to identify the time profile for the patient. This enables the patient to identify a passage of time during the medical imaging examination and hence advantageously to estimate approximately how much longer the medical imaging examination will last.

In a development of the invention, the time profile of the output information is at least partially different from a time profile of the established residual examination time for the medical imaging examination. The time profile of the established residual examination time for the medical imaging examination preferably includes several residual examination times established in succession in terms of time, by which the time profile of the established residual examination time for the medical imaging examination can be determined. This embodiment of the invention enables the output information to include a time buffer, which is not evident to the patient, but increases the temporal leeway for the medical imaging examination, for example if it is necessary to repeat partial scans of the medical imaging examination and/or delays occur in a partial scan, etc. The fact that the time buffer is not evident to the patient makes it possible to avoid unwanted worry or agitation on the part of the patient.

In a further embodiment of the invention, the time profile of the output information at least partially elapses more quickly than the time profile of the established residual examination time for the medical imaging examination. A time profile that at least partially elapses faster means that individual time segments of the time profile of the output information elapse more quickly than the equivalent and/or corresponding time segments of the time profile of the established residual examination time, in particular the anticipated residual examination time. For example, this can enable an existing time buffer to be cleared shortly before the end of the medical imaging examination without this being evident to the patient or even being detectable by the patient.

Alternatively or additionally, in a further embodiment of the invention the time profile of the output information at least partially elapses more slowly than the time profile of the established residual examination time for the medical imaging examination. A time profile that at least partially elapses slower means that individual time segments of the time profile of the output information elapse more slowly than the equivalent and/or corresponding time segments of the time profile of the established residual examination time, in particular the anticipated residual examination time. This can enable a time buffer for the output information to be established so that any interruptions of the medical imaging examination and/or repetitions of partial scans of the medical imaging examination are not evident to the patient from the output information, or are not even detectable by the patient. In addition, this can prevent unwanted worry and/or agitation on the part of the patient.

The generated output information can be independent of specific time information. Specific time information means time information expressed in physical units, namely in seconds, minutes, hours etc. The avoidance of specific time information in physical units enables the output information to deviate from the established residual examination time, in particular the anticipated residual examination time, without this being detectable by the patient or evident to the patient. A time profile of the output information can deviate from a time profile of the established residual examination time, in particular the anticipated residual examination time, without thereby attracting the patient's attention.

In a further embodiment of the invention, the generated output information is in symbol form for the residual examination time for the medical imaging examination. Output information in symbol form means that the output information does not include specific time information. The output information in symbol form can be in different symbol forms, in particular symbolizing a time profile of the medical imaging examination for the patient, for different time phases of the medical imaging examination. This has the advantage that it is possible to provide a simple acquisition of time information for the patient without this necessitating the patient having to read or acquire numbers. The output symbols, and hence the informative value of the output information, can be understandable independently of a patient's level of education, and hence will be understandable for patients who have difficulty reading.

The different output symbols preferably include mutually dependent symbols so that a time profile of the medical imaging examination can be notified to the patient using the mutually dependent symbols. For example, the mutually dependent output symbols can be different, mutually coordinated tone sequences of acoustic output information. Alternatively, the mutually dependent output symbols can be a mutually coordinated series of images, such as, for example, successive lifetime cycles of a tree. The patient is familiarized with the different output symbols before the start of the medical imaging examination so that the patient is able to categorize the output information that is provided, in particular the output symbols, in a temporal context. This enables a simple understanding of time information, in particular the time profile, without the patient having to read or acquire numbers. In addition, the output information and hence the informative value of the output information, in particular the output symbols, can be acquired independently of a patient's level of education and hence can be no problem to acquire even for patients who have difficulty reading. In addition, the output information can be advantageously acquired by patients with language barriers, such as patients who speak a different language than the medical operator.

In an embodiment of the invention, the output information that is generated and provided as an output is acoustic output information that can be detected by acoustic and/or auditory perception on the part of the patient. The acoustic output information can be, for example, a voice output to the patient. Alternatively or additionally the acoustic output information can be musical sounds and/or other types of acoustic output information that appear advisable to those skilled in the art. For example, the acoustic output information is communicated to the patient by headphones worn by the patient during the medical imaging examination.

This embodiment of the invention enables simple communication of the output information to the patient, and the acquisition of the output information is independent of the position, in particular examination position, and hence the direction of view, of the patient, and hence the output information can be acquired by the patient at any time.

Alternatively or additionally, the output information that is generated and provided as an output is optical output information that can be acquired by visual perception on the part of the patient. This enables simple perception of the output information for the patient. In addition, optical output information can advantageously divert the attention of the patient from the medical imaging examination, and hence help to relax and/or reassure the patient.

In a further embodiment of the invention, the optical output information is a display signal on a color-coded progress bar. A progress bar means an optical display object, which can be displayed on a screen and/or monitor, which indicates the progress and/or course of a procedure, in particular a time profile of the medical imaging examination, using display signals and the position thereof along the progress bar.

The different positions for display signals along the progress bar are preferably color-coded so that the signal color of a display signal alone can be used to draw conclusions with respect to the output information. In addition, a color profile of a number of display signals on the progress bar can be used to depict a time profile of the medical imaging examination for the patient. For example, the start of the progress bar and hence the start of the medical imaging examination can be given a red color. The middle of the progress bar, and hence the middle of the medical imaging examination, can then be given a yellow and/or orange color. The end of the progress bar, and hence the end of the medical imaging examination, can be given a green color. Hence, the patient can use the display symbol displayed to acquire the current status of the medical imaging examination in terms of time in a simple and quick manner.

In another embodiment of the invention, the optical output information is at least one light signal. A light signal means a light signal from a lighting unit of a patient-receiving region of the medical imaging apparatus. The light signal is preferably color-coded so that the signal color of the light signal enables conclusions to be drawn regarding the content of the output information. For example, the start of the medical imaging examination can be given a red color. A middle region in terms of time of the medical imaging examination can be given a yellow and/or orange color, for example. The end of the medical imaging examination can be given a green color, for example. Hence, the patient can use the light signal displayed to acquire the current status of the medical imaging examination in terms of time in a simple and quick manner. In addition, this enables the acquisition of the output information to be independent of the position of the patient so that preferably the lighting unit is embodied to light and/or illuminate the entire patient-receiving region and is hence evident to the patient in any desired position of the patient inside the patient-receiving region.

The optical output information can be provided as different picture symbols for different time phases of the medical imaging examination, with a second picture symbol of the different picture symbols being generated from a first picture symbol of the different picture symbols. For example, a first picture symbol can be a picture in gray tones, and, as the time profile of the medical imaging examination progresses, the subsequent picture symbols are increasingly filled with color. It is possible for individual sub-regions of the picture to be filled in succession with color tones as the medical imaging examination progresses.

In addition, it is also possible for the different picture symbols to be combined to form an animation. For example, the individual picture symbols can depict the life cycle of a tree, wherein a picture symbol with the young plant is preferably assigned to the start of the medical imaging examination and a picture symbol with the fully-grown tree is preferably assigned to the end of the medical imaging examination. The picture symbols in between are assigned to different time phases of the medical imaging examination depending on size of the tree, wherein, herein, the larger the tree depicted, the later the assigned time phase occurs within the time profile of the medical imaging examination. Alternatively, the different time phases of the medical imaging examination can be depicted by different picture symbols, wherein the different picture symbols depict a series of movements of an animal. The individual time phases of the medical imaging examination can be assigned to a specific movement status of the animal. This embodiment of the invention enables the intuitive and fast acquisition of the current time phase of the medical imaging examination without this necessitating the patient having to read or acquire numbers. In addition, the output information, and hence the informative value of the output information, can be provided independently of the patient's level of education and also to patients who have difficulty reading. In addition, the output information can also be advantageously acquired by patients with language barriers, such as patients who speak a different language than the medical operator.

The invention also concerns on a medical imaging apparatus designed to carry out a method for providing output information during a medical imaging examination, wherein the output information indicates the residual examination time for the medical imaging examination for a patient. The apparatus includes a time-acquisition processor that acquires at least one item of time information for the medical imaging examination, a control computer that establishes the residual examination time for the medical imaging examination using the time information and generates the output information as a function of the residual examination time, and an output unit that provides the generated output information to the patient in a form and at a location relative to the patient undergoing the examination so that the output information are perceptible to the user.

The invention enables communication of information to the patient during the medical imaging examination, without necessitating an interruption to the medical imaging examination. This in turn results in timesaving communication of information to the patient during the medical imaging examination. In addition, the output information during the medical imaging examination can provide a temporal pointer for categorizing the current situation in terms of time. This helps to relax the patient so that delays and/or repetitions of individual scans or partial scans of the medical imaging examination due a nervous patient can be avoided. The method for providing the output information for the medical imaging examination is preferably performed automatically and/or autonomously by means of the control computer of the medical imaging apparatus. This has the advantage of enabling a particularly timesaving examination procedure to be achieved.

The advantages of the medical imaging apparatus according to the invention substantially correspond to the advantages of the method according to the invention as described above in detail. Features, advantages and alternative embodiments noted above are also applicable to the apparatus.

The present invention also encompasses a non-transitory, computer-readable data storage medium encoded with programming instructions that, when the storage medium is loaded into a computer or computer system of a medical imaging apparatus, cause the computer or computer system to operate the medical imaging apparatus in order to implement any or all embodiments of the method according to the invention, as described above.

### BRIEF DESCRIPTION OF THE DRAWINGS

FIG. 1 is a schematic depiction of a medical imaging apparatus according to the invention.

FIG. 2 is a flowchart of the method according to the invention for providing output information during a medical imaging examination, wherein the output information indicates the residual examination time for the medical imaging examination for a patient.

FIG. 3 shows a first exemplary embodiment of the output information.

FIG. 4 shows a second exemplary embodiment of the output information.

### DESCRIPTION OF THE PREFERRED EMBODIMENTS

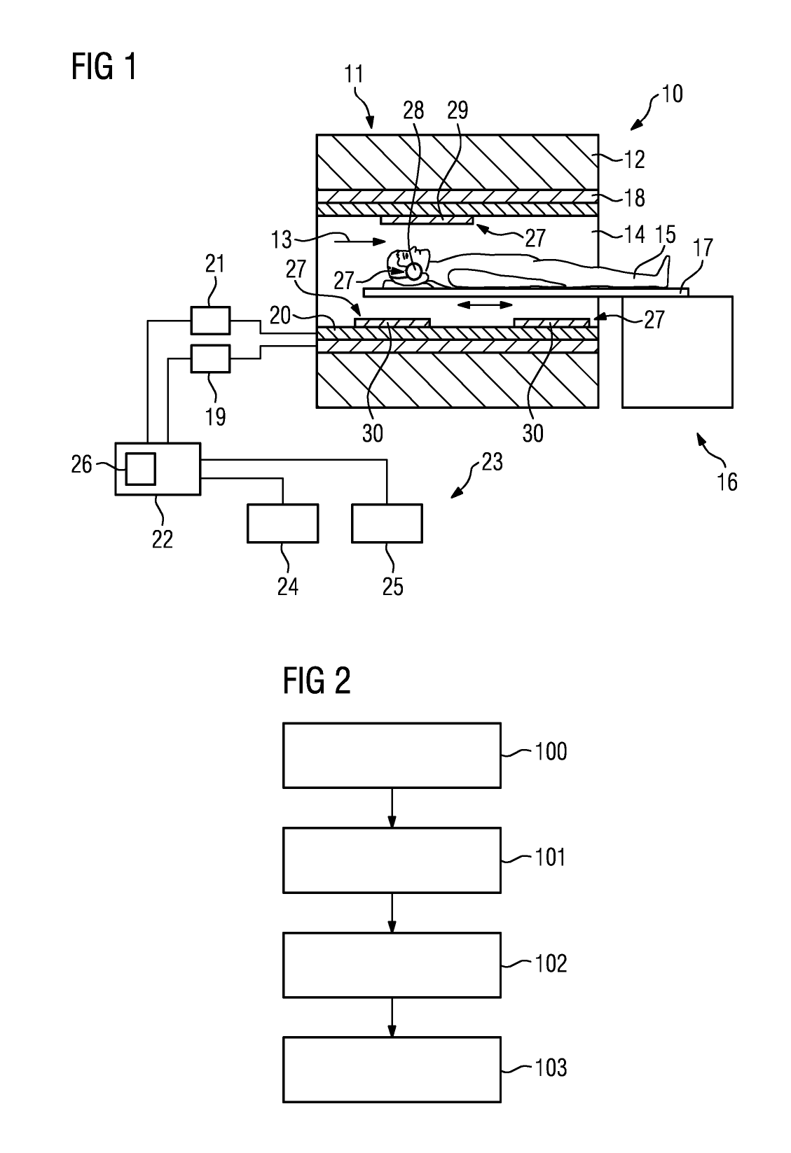

FIG. 1 is a schematic depiction of a medical imaging apparatus. In the present exemplary embodiment, the medical imaging apparatus is a magnetic resonance apparatus 10, so the present invention will be explained with the example of the magnetic resonance apparatus 10. However, the present invention is not restricted to an embodiment of the medical imaging apparatus as a magnetic resonance apparatus 10 and further embodiments of the medical imaging apparatus are conceivable, such as an embodiment of the medical imaging apparatus as a computed tomography apparatus or as a PET apparatus.

The magnetic resonance apparatus 10 has a scanner 11 with a superconducting basic field magnet 12 that generates a strong, constant, basic magnetic field 13. The scanner 11 also has a patient-receiving region 14 for receiving a patient 15. In the present exemplary embodiment, the patient-receiving region 14 has a cylindrical shape and is surrounded circumferentially by the scanner 11. Embodiments of the patient-receiving region 14 deviating from a closed cylinder is conceivable. The patient 15 can be moved into the patient-receiving region 14 by a patient-support 16 of the magnetic resonance device 10. The patient-support 16 has a patient table 17 that is movable inside the patient-receiving region 14.

The scanner 11 further has a gradient coil arrangement 18 that generates magnetic field gradients, which are used for spatially encoding MR signals during imaging. The gradient coil arrangement 18 is controlled by a gradient controller 19. The scanner 11 further has a radio-frequency (RF) antenna unit 20 controlled by a radio-frequency controller 21 so as to emit radio-frequency magnetic resonance sequences in an examination volume substantially formed by the patient-receiving region 14 of the scanner 11. The radiated RF sequences give certain nuclear spins in the patient 15 a magnetization that causes those nuclear spins to deviate from the basic magnetic field 13 by an amount known as a flip angle. As these excited nuclear spins relax and return to the steady state, they emit the aforementioned MR signals, which are also RF signals, and which may be detected by the same antenna that radiated the RF sequence, or by a different antenna.

To control the basic field magnet 12, the gradient controller 19, the radio-frequency controller 21, the magnetic resonance device 10 has a system control computer 22. The system control computer 22 controls the magnetic resonance apparatus 19 centrally, such as for the performance of a predetermined imaging gradient echo sequence. The system control computer 22 also has an evaluation processor for the evaluation of medical image data acquired during the magnetic resonance examination.

The magnetic resonance apparatus 10 also has a user interface 23 connected to the system control computer 22. Control information such as imaging parameters, and reconstructed magnetic resonance images, can be displayed on a display unit 24, for example on at least one monitor and/or screen, of the user interface 23 for a medical operator. The user interface 23 also has an input unit 25 via which the medical operator can enter information and/or parameters during a scanning process.

FIG. 2 depicts the method according to the invention for providing output information during a medical imaging examination, wherein the output information indicates the residual examination time for the medical imaging examination for the patient 15. Herein, the control computer 22 is provided to implement the method automatically and/or autonomously.

In order to implement the method the control computer 22 has computer programs and/or software, which can be loaded directly in a memory, with program code that cause the method to be implemented when the computer code is executed in the control computer 22. To this end, the control computer 22 has a core processor and the memory, in which the software and/or computer programs are stored.

The software and/or computer programs can be stored on an electronically readable data storage medium embodied separately from the control computer 22 and/or separately from the magnetic resonance apparatus 10, so the storage medium can be loaded into the computer 22.

In a first method step 100 of the method, at least one item of time information for the medical imaging examination, in particular the magnetic resonance examination, is acquired. The acquisition of the at least one item of time information is preferably performed by means of a time-acquisition processor 26 of the medical imaging apparatus, in particular the magnetic resonance apparatus 10. In the present exemplary embodiment, the time-acquisition processor 26 is part of the control computer 22 of the magnetic resonance apparatus 10. Alternatively, the time-acquisition processor 26 can be embodied separately from the control computer 22 and/or from magnetic resonance apparatus 10. The time information is preferably acquired automatically and/or autonomously by the time-acquisition processor 26. The time-acquisition processor 26 can be, for example, a clock.

The established time information for the medical imaging examination, in particular the magnetic resonance examination, preferably is a duration of the medical imaging examination, from the beginning of the medical imaging examination up to a time point of the acquisition of the time information for the medical imaging examination.

In a subsequent method step 101, the residual examination time, in particular an anticipated residual examination time, is established for the medical imaging examination using the acquired time information for the medical imaging examination. The establishment of the residual examination time, in particular the anticipated residual examination time, for the medical imaging examination is performed by the control computer 22 of the medical imaging apparatus 10. To this end, the control computer 22 preferably executes an evaluation algorithm and/or a determining algorithm.

The establishment of the residual examination time, in particular the anticipated residual examination time, for the medical imaging examination is preferably performed automatically and/or autonomously by the control computer 22.

In this method step 101, the residual examination time, in particular the anticipated residual examination time, for the medical imaging examination is established in comparison to a total duration of the medical imaging examination by the control computer 22. The total duration of the magnetic resonance examination, preferably is a time interval from the beginning of the magnetic resonance examination, to its end, in particular a planned end.

The total duration of the magnetic resonance examination, is established and/or calculated by the control computer 22 using settings for the magnetic resonance examination, in the method step 101. The settings of the magnetic resonance examination, can be, for example, an examination region of the patient 15, such as the head or a knee or the abdomen, etc. The settings of the magnetic resonance examination, can also be dependent on a medical or diagnostic issue. The settings of the magnetic resonance examination can also be parameter settings that can influence the duration of the magnetic resonance examination. For example, the parameter settings can be an echo time and/or a slice thickness, etc.

In the method step 101, the total duration of the magnetic resonance examination is also established using an empirical value for the selected magnetic resonance examination, by the control computer 22. The empirical value for the magnetic resonance examination can be notified by a medical operator carrying out the magnetic resonance examination, via the input unit 25 of the user interface 23 of the control computer 22. For example, the total duration of an abdominal examination on a patient 15 can last several minutes longer than was established by the control computer 22 using only the settings of the magnetic resonance examination. Delays to the total duration calculated using only the settings of the magnetic resonance examination can be caused by breathing instructions to the patient 15 and/or changes in the position of the patient table 17 and/or pauses for contrast agent accumulations, which were not taken into account during a calculation of the total duration using only the settings. In addition, in the case of breathing-trigged magnetic resonance examinations, the total duration of the magnetic resonance examination is also dependent upon the breathing rate and/or breathing amplitude of the patient 15, which as such are not taken into account in a calculation of the total duration by the control computer 22 using only the settings of the medical imaging examination.

Providing an input of an empirical value during the establishment and/or calculation of the total duration of the magnetic resonance examination in the method step 101 can also be useful if the magnetic resonance examination has already started but the planning of the magnetic resonance examination has not yet been fully completed.

In a further method step 102, the output information is generated as a function of the residual examination time, in particular the anticipated residual examination time, for the magnetic resonance examination, by the control computer 22. To this end, the control computer 22 executes an evaluation algorithm and/or a determining algorithm. The generation of the output information as a function of the residual examination time, in particular the anticipated residual examination time, for the medical imaging examination is preferably performed automatically and/or autonomously by the control computer 22.

In this method step 102, a second anticipated residual examination time determined for the output information by the control computer 22. The second anticipated residual examination time determined for the output information can differ from the residual examination time determined in the previous method step. For example, a time buffer, that takes account of the upcoming scans of the magnetic resonance examination can be taken into account during the determination and/or establishment of the second, anticipated residual examination time. The second anticipated residual examination time determined for the output information is used to generate the output information.

Preferably, during the magnetic resonance examination, several items of output information are generated by the control computer 22. The several items of output information are generated at different time points of the magnetic resonance examination. In addition, output information can be generated continuously during the magnetic resonance examination by the control computer 22, as long as time information is acquired continuously in the method step 100.

The control computer 22 uses the several items of output information to generate or establish a time profile for the patient 15. Due to the generation of the output information using the second, anticipated residual examination time determined for the output information, the time profile of the output information can be at least partially different from a time profile of the established residual examination time, in particular the anticipated residual examination time, for the magnetic resonance examination. The time profile of the established residual examination time for the medical imaging examination preferably includes a number of residual examination times established in succession in terms of time. This enables the output information to include a time buffer, which is advantageously not evident to the patient 15 but which increases the temporal leeway for the magnetic resonance examination, for example if it is necessary to repeat partial scans of the medical imaging examination, in particular the magnetic resonance examination, and/or if delays occur during a partial scan etc.

The time profile of the output information can at least partially elapse more slowly than the time profile of the established residual examination time, in particular the anticipated residual examination time, for the magnetic resonance examination. This enables an advantageous establishment of a time buffer for the output information, so that any interruptions of the magnetic resonance examination, and/or repetitions of partial scans of the magnetic resonance examination, are not evident to the patient from the output information.

In addition, the time profile of the output information can also at least partially elapse more quickly than the time profile of the established residual examination time, in particular the anticipated residual examination time, for the magnetic resonance examination. This can be advantageous shortly before the end of the magnetic resonance examination, since this enables any time buffer still present to be cleared.

In the further method step 102, the output information is generated by the control computer 22 independently of a specific time. Hence, the generated output information does not have any specific time information in physical units, such as, for example, in seconds, minutes, hours etc., so that the patient 15 cannot identify from the output information whether the time profile of the output information is elapsing more quickly or more slowly, and hence the output information does not cause additional worry to the patient 15.

The output information generated by the control computer 22 can be in symbol form for the residual examination time, in particular the anticipated residual examination time, for the magnetic resonance examination. The output information in symbol form can symbolically depict, for example, different time phases of the magnetic resonance examination, comprise different output information in symbol form, so as to symbolize a time profile of the magnetic resonance examination, for the patient 15.

The different output symbols generated by the control computer 22 preferably are mutually dependent symbols that enable a simple depiction of a time profile for the patient 15. For example, the mutually dependent output symbols can be different tone sequences of acoustic output information. Alternatively, the mutually dependent output symbols can be a mutually coordinated series of images, such as successive life cycles of a tree.

Before the start of the method for providing the output information for the medical imaging examination and/or before the start of the medical imaging examination, the patient 15 is familiarized with the different output symbols so that the patient able to categorize the acquired output information acquired, in particular the output symbols, in a temporal context.

In a subsequent method step 103, the output information is provided to the patient 15. First, the generated output information is transferred by the control computer 22 to an output unit 27 of the magnetic resonance device 10 via a data transfer unit of the magnetic resonance apparatus 10 (not shown). The output information is provided to the patient 15 by the output unit 27, with the output unit 27 being part of the magnetic resonance apparatus 10. In the exemplary embodiment, the output unit 27 is formed by an optical output unit and an acoustic output unit. In the exemplary embodiment, the acoustic output unit is headphones 28, worn by the patient 15 during the magnetic resonance examination. If the output information is acoustic output information, the headphones 28 are used to communicate the output information to the patient 15.

In the exemplary embodiment, the optical output unit is a screen 29 and/or monitor inside the patient-receiving region 14, on an opposite side of the patient-receiving region 14 to the patient 15. The optical output unit further includes a lighting unit 30 for lighting and/or illuminating the patient-receiving region 14. The lighting unit 30 is preferably designed to emit light signals with different wavelengths and hence also light with different colors. If the output information includes optical output information, the screen 29 and/or monitor and/or the lighting unit 30 are used to communicate the output information to the patient 15.

Alternatively, the optical output unit can be video glasses and/or a projection unit with which optical information can be projected from outside of the patient-receiving region 14 onto a projection surface inside the patient-receiving region 14. The optical output unit may also be further types of optical output elements that appear advisable to those skilled in the art.

Alternatively to the present output unit 27, which includes both an optical output unit and an acoustic output unit, the output unit 27 can also have only one acoustic output unit or only one optical output unit.

The output information generated in the further method step 102 can be acoustic output information. The acoustic output information can be, for example, voice output, which can be provided to the patient 15 by the acoustic output unit, in particular the headphones 28, in the method step 103. Alternatively or additionally, the acoustic output information can be a sequence of different tones and/or musical sounds and/or further acoustic output information that appears advisable to those skilled in the art.

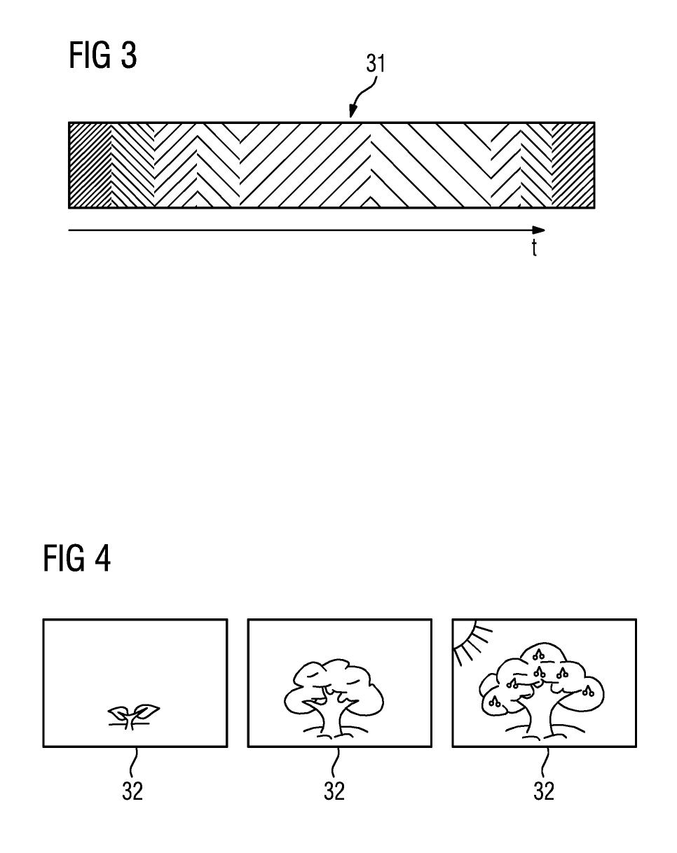

The output information generated in the further method step 102 can also be optical output information, provided by the optical output unit, in particular the screen 29 and/or the monitor and/or the lighting unit 30, to the patient 15 in the method step 103. The optical output information can be, for example, a display signal on a color-coded progress bar 31 as depicted in FIG. 3, a first exemplary embodiment of optical output information. The optical output information is displayed by a position and a color of display signals on the progress bar 31. The different positions for display signals along the progress bar 31 are color-coded so that the signal color of a display signal alone can be used to draw conclusions with respect to the content of the output information.

In addition, a color profile of a number of display signals on the progress bar 31 can be used to depict a time profile for the patient 15. For example, the start of the progress bar 31, and hence the start of the magnetic resonance examination, can be given a red color. In addition, the middle region of the progress bar 31, and hence the middle of the magnetic resonance examination can be given a yellow and/or orange color, for example. The end of the progress bar 31, and hence the end of the magnetic resonance examination can be given a green color, for example.

In addition to the embodiment of the optical output information as a display signal on a color-coded progress bar 31, the optical output information can also be at least one light signal, which can be provided to the patient 15 by the lighting unit 30. The light signal is preferably color-coded so that conclusions with respect to the content of the output information can be drawn from the signal color of the light signal. For example, the start of the magnetic resonance examination can be given a red color. A middle region in terms of time of the magnetic resonance examination can be given a yellow and/or orange color, for example. The end of the magnetic resonance examination can be given a green color, for example.

The optical output information can be different picture symbols 32 for different time phases of the magnetic resonance examination, wherein a second picture symbol 32 of the different picture symbols 32 is generated from a first picture symbol 32 of the different picture symbols 32. For example, a first picture symbol 32 can be a picture in gray tones, and, as the time profile of the medical imaging examination progresses, the following picture symbols 32 are increasingly filled with color. It is possible for individual sub-regions of the picture to be filled in succession with color tones as the medical imaging examination progresses.

It is also possible for the different picture symbols 32 to be combined to form an animation. For example, the individual picture symbols 32 can depict the life cycle of a tree as depicted in FIG. 4, a further exemplary embodiment of the output information. The picture symbol 32 with the young plant is preferably assigned to the start of the magnetic resonance examination, and the picture symbol 32 with the fully-grown tree assigned to the end of the magnetic resonance examination. The picture symbols 32 in between are assigned to different time phases of the magnetic resonance examination, depending on the size of the tree. The larger the tree depicted, the later the assigned time phase occurs within the time profile of the magnetic resonance examination.

Alternatively, the different time phases of the magnetic resonance examination can be depicted by picture symbols 32, wherein the different picture symbols 32 depict a series of movements of an animal. The individual time phases of the magnetic resonance examination can be assigned to a specific movement status of the animal. In addition, further picture symbols 32 that appear advisable to those skilled in the art are conceivable.

Although modifications and changes may be suggested by those skilled in the art, it is the intention of the Applicant to embody within the patent warranted hereon all changes and modifications as reasonably and properly come within the scope of the Applicant's contribution to the art.

1. A method for providing information to a patient during a medical imaging examination, said method comprising:
operating a medical imaging apparatus, while a patient is undergoing an examination with the medical imaging apparatus;
during said examination, providing a computer with time information that informs said computer as to an amount of time that has elapsed from a beginning of said examination;
from said time information, determining, in said computer, a residual examination time for the examination that designates an ending time at which said examination is expected to end;
generating output information in said computer dependent on said residual examination time, said output information comprising humanly perceptible information formatted to inform the patient as to when said examination is expected to end; and
providing said output information in said humanly perceptible form to the patient while the patient is undergoing said examination.

2. A method as claimed in claim 1 comprising providing said time information to said computer that represents a duration of the examination from the start of the examination until an acquisition of at least one item of time information for the examination.

3. A method as claimed in claim 1 comprising establishing said residual examination time in said computer in comparison to a total expected duration of said examination.

4. A method as claimed in claim 3 comprising calculating said total expected duration of said examination using settings provided to the computer for the examination.

5. A method as claimed in claim 3 comprising determining said total duration of the examination as a function of an empirical value obtained and provided to the computer during said examination.

6. A method as claimed in claim 1 comprising generating a plurality of items of output information during said examination, from which a time profile is created in said computer, and providing said time profile to the patient in said output information.

7. A method as claimed in claim 6 comprising creating said time profile so as to be different from the residual examination time for the examination.

8. A method as claimed in claim 6 comprising creating said time profile so that said time profile elapses more quickly than a time profile of said residual examination time.

9. A method as claimed in claim 6 comprising creating said time profile so that said time profile elapses more slowly than a time profile of said residual examination time.

10. A method as claimed in claim 1 comprising generating said output information in said computer independently of a specific time.

11. A method as claimed in claim 1 comprising generating said output information in symbolic form.

12. A method as claimed in claim 1 comprising generating said output information as a plurality of different output symbols for different time phases of the examination.

13. A method as claimed in claim 12 wherein said different output symbols are mutually dependent on each other.

14. A method as claimed in claim 1 comprising generating said output information as acoustic output information.

15. A method as claimed in claim 1 comprising generating said output information as optical output information.

16. A method as claimed in claim 15 comprising generating said optical output information as a display signal on a color-coded progress bar at a display operated by said computer.

17. A method as claimed in claim 15 wherein said medical imaging apparatus comprises a light source that emits a light signal, and comprising controlling said light source dependent on said output information to embody said output information in said light signal.

18. A method as claimed in claim 15 comprising generating said optical output information as a plurality of different picture symbols for different time phases of the examination, in a sequence comprising at least a first picture symbol and a second picture symbol, with said second picture symbol being generated from said first picture symbol.

19. A medical imaging apparatus comprising:
a medical image data acquisition unit;
a computer configured to operate the medical image data acquisition unit while a patient is undergoing an examination with the medical image data acquisition unit;
during said examination, said computer being configured to receive time information that informs said computer as to an amount of time that has elapsed from a beginning of said examination;
said computer being configured to determine, from said time information, a residual examination time for the examination that designates an ending time at which said examination is expected to end;
said computer being configured to generate output information dependent on said residual examination time, said output information comprising humanly perceptible information formatted to inform the patient as to when said examination is expected to end; and
an output unit in communication with said computer configured to provide said output information in said humanly perceptible form to the patient while the patient is undergoing said examination.

20. A non-transitory, computer-readable data storage medium encoded with programming instructions, said storage medium being loaded into a computer system of a medical imaging apparatus and said programming instructions causing said computer system to:
operate the medical imaging apparatus, while a patient is undergoing an examination with the medical imaging apparatus;
during said examination, receive time information that informs said computer system as to an amount of time that has elapsed from a beginning of said examination;
from said time information, determine a residual examination time for the examination that designates an ending time at which said examination is expected to end;
generate output information dependent on said residual examination time, said output information comprising humanly perceptible information formatted to inform the patient as to when said examination is expected to end; and
provide said output information in said humanly perceptible form to the patient while the patient is undergoing said examination.
